(19) United States
(12) Patent Application Publication
(10) Pub. No.: US 2025/0305996 A1
(43) Pub. Date: Oct. 2, 2025

(54) NON-TRANSITORY COMPUTER-READABLE RECORDING MEDIUM, FRESHNESS DETERMINATION METHOD, AND FRESHNESS DETERMINATION APPARATUS

(71) Applicants: Fujitsu Limited, Kawasaki-shi (JP); TOKAI UNIVERSITY EDUCATIONAL SYSTEM, Tokyo (JP)

(72) Inventors: Akira SAKAI, Kawasaki (JP); Suguru YASUTOMI, Kawasaki (JP); Keiichi GOTO, Shizuoka (JP); Masafumi YAGI, Shizuoka (JP); Kazuki MIZUNO, Shizuoka (JP)

(73) Assignees: Fujitsu Limited, Kawasaki-shi (JP); TOKAI UNIVERSITY EDUCATIONAL SYSTEM, Tokyo (JP)

(21) Appl. No.: 19/234,370

(22) Filed: Jun. 11, 2025

Related U.S. Application Data
(63) Continuation of application No. PCT/JP2022/046022, filed on Dec. 14, 2022.

(51) Int. Cl. G01N 29/44; G01N 29/07; G01N 33/12

(52) U.S. Cl. CPC G01N 29/4472; G01N 29/07; G01N 33/12; G01N 2291/011; G01N 2291/023

(57) ABSTRACT

A freshness determination program, a freshness determination method, and a freshness determination apparatus that nondestructively determine the freshness of a frozen target object accurately are provided. A computer is caused to execute processing of acquiring waveform data of an ultrasonic wave with a frequency of 1 MHz or less that is propagated in a frozen target object, inputting the waveform data to a machine training model, and determining the freshness of a target object.

### CROSS-REFERENCE TO RELATED APPLICATION

This application is a continuation application of International Application PCT/JP2022/046022, filed on Dec. 14, 2022, and designating the U.S., the entire contents of which are incorporated herein by reference.

### FIELD

The present invention relates to a freshness determination program, a freshness determination method, and a freshness determination apparatus.

### BACKGROUND

As one of information indicating the quality of fish such as a tuna, there is an index called freshness. Here, the freshness indicates a degree of postmortem rigidity. For example, the freshness of a frozen tuna varies depending on a timing at which a tuna has started to be frozen, the state of a freezer, and the like.

To detect the freshness of fish, an inspection that uses freshness test paper, the analysis of adenosine triphosphate (ATP) by chromatography, the inspection of cross-section by tail cutting screening, and the like have been conventionally executed. Nevertheless, these methods are invasive for a targeted frozen tuna, and maintenance the commercial value of a tuna has become an issue. Further, because a frozen tuna is hard, a dedicated device is used for fabricating a frozen tuna, and there is a concern that significant damage occurs during fabrication. Thus, in the invasive inspection methods, difficulty in fabrication of the frozen tuna has become a problem. Note that, as a technique of fish quality evaluation that uses ultrasonic waves, there has been proposed a technique of performing a nondestructive inspection of fresh fish using a medical ultrasonic device with a frequency band from 1 MHz to 100 MHz.
Patent Literature 1: Japanese Laid-open Patent Publication No. 2007-155692

### Non Patent Literature

Non Patent Literature 1: “Fish freshness determination method and measurement accuracy of FTP method”, Shigeo Ehira, Hitoshi Uchiyama, [online], Apr. 26, 2013, Science of Cookery, [Search on Nov. 16, 2022], Internet <URL:.jstage.jst.go.jp/article/cookerysciencel968/19/3/19_176/_pdf/-char/ja>
Non Patent Literature 2: “Fish freshness (K-value) test method—high-performance liquid chromatographic method”, [online], Mar. 31, 2022, Ministry of Agriculture, Forestry and Fisheries of Japan, [Search on Nov. 16, 2022], Internet <URL:.maff.go.jp/j/jas/jas_kikaku/attach/pdf/kokujikaisei-230.pdf>

### SUMMARY

According to an aspect of an embodiment, a non-transitory computer-readable recording medium stores therein a freshness determination program that causes a computer to execute a process including acquiring waveform data of an ultrasonic wave with a frequency of 1 MHz or less that is propagated in a frozen target object, and determining freshness of the target object by inputting the waveform data to a machine training model.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1 is a schematic configuration diagram illustrating a freshness determination system according to an embodiment.

FIG. 2 is a block diagram of a freshness determination apparatus according to an embodiment.

FIG. 3 is a diagram illustrating an example of a measurement position of a beam line to be used in freshness determination.

FIG. 4 is a diagram illustrating an example of a measurement position designation screen.

FIG. 5 is a diagram illustrating comparison between a waveform of a normal frozen tuna and a waveform of a frozen tuna with poor freshness.

FIG. 6 is a diagram illustrating an example of a power spectrum.

FIG. 7 is a diagram illustrating an example of a measurement in-execution screen.

FIG. 8 is a diagram illustrating an example of an overall result display screen.

FIG. 9A is a first diagram illustrating a representative example of waveform data of a reflective wave obtained from a normal frozen tuna.

FIG. 9B is a second diagram illustrating a representative example of waveform data of a reflective wave obtained from a normal frozen tuna.

FIG. 10A is a first diagram illustrating a representative example of waveform data of a reflective wave obtained from a frozen tuna with poor freshness.

FIG. 10B is a second diagram illustrating a representative example of waveform data of a reflective wave obtained from a frozen tuna with poor freshness.

FIG. 11 is a flowchart of freshness determination processing according to an embodiment.

FIG. 12 is a hardware configuration diagram.

### DESCRIPTION OF EMBODIMENTS

Nevertheless, in a case where ultrasonic waves in a frequency band of 1 MHz or more are propagated in a frozen tuna, attenuation of nearly 10 dB per cm occurs. Thus, in a frozen tuna inspection that uses ultrasonic waves with the frequency band of 1 MHz or more, because an attenuation coefficient is very large and it is difficult to obtain waveform data of appropriate reflective waves, it is difficult to perform a nondestructive inspection of a frozen tuna. In this manner, in the conventional technique, it has been difficult to nondestructively determine the freshness of a frozen target object.

Hereinafter, an embodiment of a freshness determination program, a freshness determination method, and a freshness determination apparatus disclosed by the present application will be described in detail based on the drawings. Note that, a freshness determination method, and a freshness determination apparatus disclosed by the present application are not limited by the following embodiment.

### (a) First Embodiment

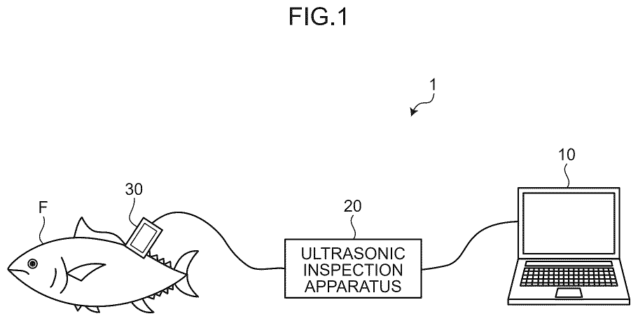

FIG. 1 is a schematic configuration diagram illustrating a freshness determination system according to an embodiment. A freshness determination system 1 includes a freshness determination apparatus 10, an ultrasonic inspection apparatus 20, and an ultrasonic probe 30. The freshness determination system 1 determines the freshness of a frozen target object. The target object includes fish and edible fish meat in which postmortem rigidity occurs, for example. In the present embodiment, the freshness determination system 1 determines the freshness of a frozen tuna F as a frozen target object. The freshness determination apparatus 10 is connected to the ultrasonic inspection apparatus 20.

The ultrasonic probe 30 is a linear array probe in which an ultrasonic wave irradiation part and a reflective wave receiving part are arranged at the same location. The ultrasonic probe 30 receives information regarding ultrasonic irradiation settings such as frequency, power, and directions from the ultrasonic inspection apparatus 20. Then, the ultrasonic probe 30 performs scanning of a target object using echography of emitting ultrasonic waves to the frozen tuna F in accordance with a designated irradiation setting, and receiving again ultrasonic waves reflected by the frozen tuna F.

The ultrasonic inspection apparatus 20 receives an ultrasonic wave transmission instruction from the freshness determination apparatus 10. Then, the ultrasonic inspection apparatus 20 transmits information regarding ultrasonic irradiation settings such as frequency, power, and directions to the ultrasonic probe 30, causes the ultrasonic probe 30 to emit ultrasonic waves to the frozen tuna F, and acquires a reflected signal of ultrasonic waves propagated in the frozen tuna F, using the ultrasonic probe 30. Here, in the freshness determination system 1 according to the present embodiment, because the scanning of a frozen fish such as the frozen tuna F is performed, the ultrasonic inspection apparatus 20 outputs ultrasonic waves in a low frequency band from 20 kHz to 1 MHz. The ultrasonic inspection apparatus 20 transmits data of reflective waves of ultrasonic waves obtained by the ultrasonic probe 30, to the freshness determination apparatus 10.

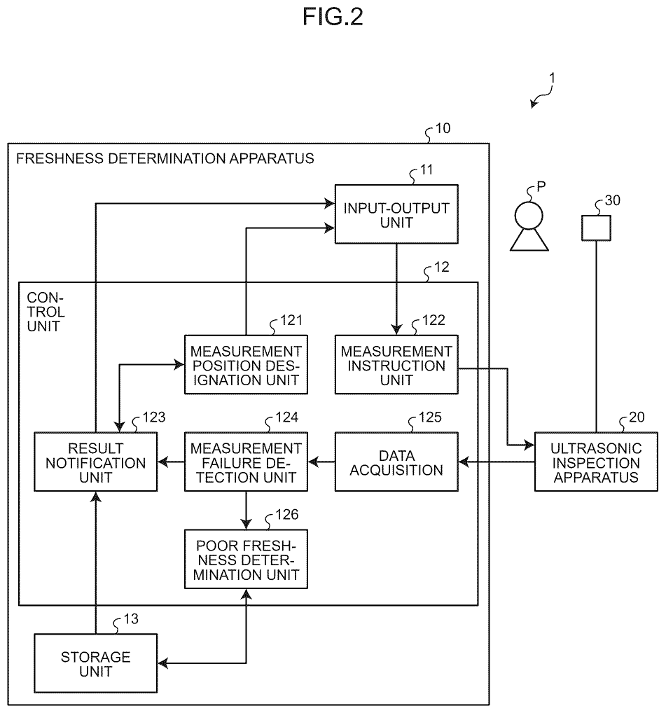

The freshness determination apparatus 10 transmits an ultrasonic wave transmission instruction to the ultrasonic inspection apparatus 20, and receives data of reflective wave obtained by the scanning of the frozen tuna F, from the ultrasonic inspection apparatus 20. Then, the freshness determination apparatus 10 inputs waveform data obtained from the acquired data of reflective wave, to a trained machine training model, and performs the determination of poor freshness at a measurement position in the frozen tuna F. After that, the freshness determination apparatus 10 notifies a user P of a final determination result of freshness of the frozen tuna F. The user P is a buyer of frozen tunas, for example. Hereinafter, the details of the freshness determination apparatus 10 will be described. As illustrated in FIG. 2, the freshness determination apparatus 10 includes an input-output unit 11, a control unit 12, and a storage unit 13.

The input-output unit 11 includes an output device such as a monitor, and an input device such as a keyboard and a mouse. The input-output unit 11 receives an instruction from the control unit 12, and displays a designated screen on the output device. Further, the input-output unit 11 outputs data and commands input by the user P using the input device, to the control unit 12.

The control unit 12 issues an ultrasonic wave transmission instruction to the ultrasonic inspection apparatus 20. Then, the control unit 12 receives, from the ultrasonic inspection apparatus 20, data of reflective waves of ultrasonic waves with a frequency of 500 kHz that have been emitted to the frozen tuna F, for example, and determines the freshness of the frozen tuna F using waveform data of reflective waves. As illustrated in FIG. 2, the control unit 12 includes a measurement position designation unit 121, a measurement instruction unit 122, a result notification unit 123, a measurement failure detection unit 124, a data acquisition 125, and a poor freshness determination unit 126. The ultrasonic wave with the frequency of 500 kHz has been used as an example, but the frequency is not limited as long as the frequency falls within the range from 20 kHz to 1 MHz, and ultrasonic waves with a plurality of frequencies may be used.

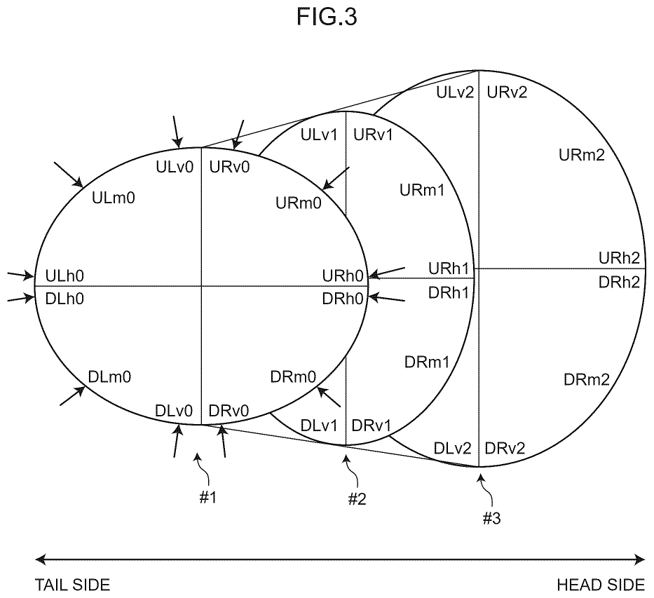

The measurement position designation unit 121 preliminarily holds information regarding a measurement position in the frozen tuna F where ultrasonic waves are emitted. FIG. 3 is a diagram illustrating an example of a measurement position of to be used in freshness determination. FIG. 3 schematically illustrates three cross-sections #1 to #3 of the frozen tuna F with a back fin oriented upward. The cross-section #1 is a tail side of the frozen tuna F, and the cross-section #3 is a head side of the frozen tuna F. Then, in each of the cross-sections #1 to #3, a measurement position with which the ultrasonic probe 30 is brought into contact is designated. For example, in the case of the cross-section #1, ULv0, ULm0, ULh0, URv0, URm0, URh0, DLv0, DLm0, DLh0, DRv0, DRm0, and DRh0 denote measurement positions with which the ultrasonic probe 30 is brought into contact. In FIG. 3, the measurement positions are indicated while assuming that an upside viewed from the tail side of the frozen tuna F is denoted by “U”, and a downside is denoted by “D”. Further, the measurement positions are indicated while assuming that the right side viewed from the tail side of the frozen tuna F is denoted by “R”, and the left side is denoted by “L”. Further, positions of beam lines are indicated assuming that a vertical direction of the frozen tuna F is denoted by “v”, the middle is denoted by “m”, and a horizontal direction is denoted by “h”. For example, URh0 denotes a measurement position corresponding to irradiation from upper and rightward horizontal direction viewed from the tail side in the cross-section #1 on the tail side of the frozen tuna F. Here, a direction from the head toward the tail of the frozen tuna F will be referred to as a front-back direction, and a direction from the back fin to a pectoral fin will be referred to as an up-down direction.

The measurement position designation unit 121 holds, for example, ULv0 to ULv2, ULm0 to ULm2, ULh0 to ULh2, URv0 to URv2, URm0 to URm2, URh0 to URh2, DLv0 to DLv2, DLm0 to DLm2, DLh0 to DLh2, DRv0 to DRv2, DRm0 to DRm2, and DRh0 to DRh2 illustrated in FIG. 3, as measurement positions. It is preferable that measurement positions are provided at four points in total for each surface of the frozen tuna F, including at least two points arranged in the front-back direction that are arranged on two lines in the up-down direction. Further, measurement positions preferably include a position near the tail of the frozen tuna F, or includes at least a position closer to the tail side than the center in the front-back direction.

Further, the measurement position designation unit 121 has a selection algorithm of selecting all measurement positions by sequentially selecting a measurement position from among held measurement positions one by one. The selection algorithm repeats, for example, selecting measurement positions one by one from the rearmost column on the uppermost row forward from among measurement positions arranged in the front-back and up-down directions of one surface of the frozen tuna F, and returning to the rearmost column on the row immediately below the uppermost row if the position reaches the foremost column, up to the foremost column on the lowermost row. If the freshness determination processing is started, the measurement position designation unit 121 determines a first measurement position to be a probe contact position from among the measurement positions in accordance with the selection algorithm.

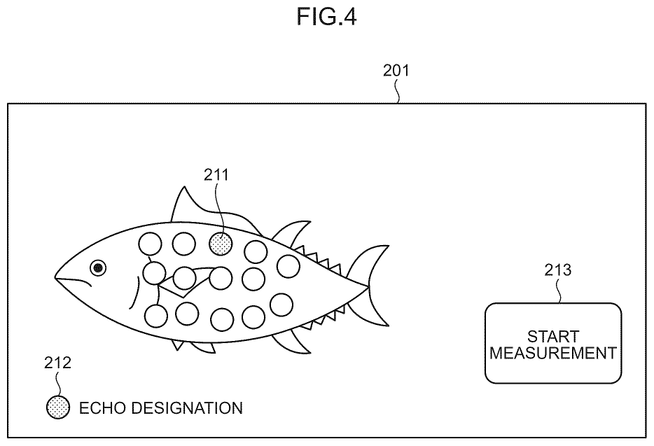

Then, the measurement position designation unit 121 displays a measurement position designation screen 201 indicating a probe contact position, as illustrated in FIG. 4, on a display device of the input-output unit 11. FIG. 4 is a diagram illustrating an example of a measurement position designation screen. The measurement position designation unit 121 thereby presents a probe contact position in the frozen tuna F with which the ultrasonic probe 30 is brought into contact, to the user P.

For example, as illustrated in FIG. 4, the measurement position designation unit 121 arranges an image of the frozen tuna F on the measurement position designation screen 201, and arranges each measurement position on the image of the frozen tuna F. Furthermore, the measurement position designation unit 121 adds a predetermined pattern to a probe contact position 211 among measurement positions arranged on the measurement position designation screen 201. Further, the measurement position designation unit 121 arranges a display example 212 of a pattern indicating a probe contact position, on the measurement position designation screen 201. Moreover, the measurement position designation unit 121 arranges a measurement start button 213 on the measurement position designation screen 201.

The user P checks a probe contact position by referring to the measurement position designation screen 201, and brings the ultrasonic probe 30 into contact with the designated probe contact position. Then, the user P issues a measurement start instruction by pressing the measurement start button 213 provided on the measurement position designation screen 201.

If no measurement failure occurs in the scanning by the ultrasonic probe 30 after measurement is performed, the measurement position designation unit 121 receives a designation instruction of the next probe contact position from the result notification unit 123. In response to the instruction from the result notification unit 123, the measurement position designation unit 121 determines the next measurement position to be a probe contact position from among the measurement positions in accordance with the selection algorithm. Then, the measurement position designation unit 121 displays a measurement position designation screen 201 indicating the next probe contact position on the display device of the input-output unit 11.

After that, the measurement position designation unit 121 receives a notification indicating that a result notification has been made, from the result notification unit 123. Then, the measurement position designation unit 121 determines whether or not freshness determination at all measurement positions has been completed. In a case where a measurement position of which freshness determination has not been performed remains, the measurement position designation unit 121 determines that the inspection has not ended, and performs the selection of the next measurement position.

In contrast to this, in a case where determination of freshness at all measurement positions has been completed, the measurement position designation unit 121 determines that the inspection has ended. If freshness determination at all measurement positions is completed, the measurement position designation unit 121 notifies the result notification unit 123 of an inspection completion notification.

If the measurement start button 213 provided on the measurement position designation screen 201 is pressed by the user P, the measurement instruction unit 122 receives a measurement start instruction from the input-output unit 11. Then, the measurement instruction unit 122 issues an ultrasonic wave transmission instruction to the ultrasonic inspection apparatus 20.

The data acquisition 125 receives, from the ultrasonic inspection apparatus 20, data of reflective waves reflected by the frozen tuna F after ultrasonic waves with the frequency of 500 kHz are emitted from the ultrasonic probe 30. Then, the data acquisition 125 outputs the acquired data of reflective waves to the measurement failure detection unit 124.

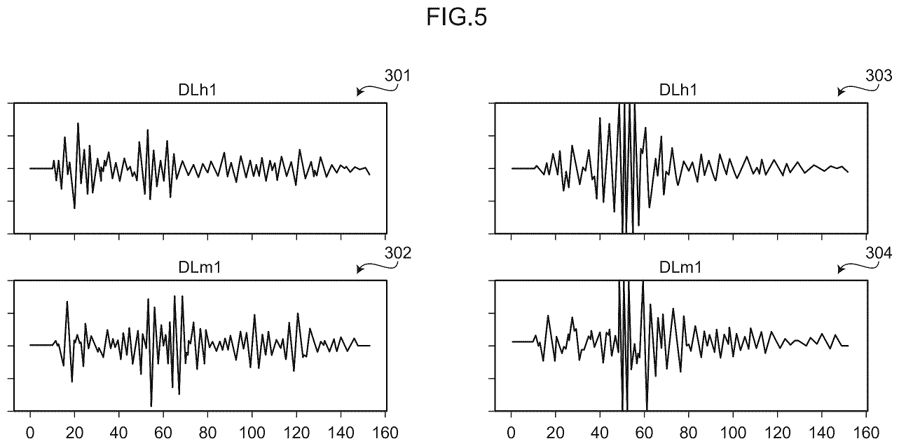

FIG. 5 is a diagram illustrating comparison between a waveform of a normal frozen tuna and a waveform of a frozen tuna with poor freshness. Graphs 301 and 302 indicate waveforms of normal frozen tunas without poor freshness. Further, graphs 303 and 304 indicate waveforms of frozen tunas with poor freshness. In the graphs 301 to 304, a horizontal axis indicates an elapsed time of transmission and reception of an ultrasonic wave, and a vertical axis indicates the amplitude of a reflective wave. The data acquisition 125 acquires data of reflective waves forming waveforms as indicated by the graphs 301 to 304, for example, and outputs the data to the measurement failure detection unit 124.

The measurement failure detection unit 124 includes a trained machine training model of outputting information regarding a measurement failure such as peel-off, dirt, or a contact failure using an input of the data of, reflective wave. Here, the “peel-off” refers to a state in which the skin of the frozen tuna F is peeled off by being pried. Further, the “dirt” refers to a state in which the frozen tuna F is soiled by blood or the like attaching thereto. Further, the “contact failure” refers to a state in which the ultrasonic probe 30 is not accurately brought into contact with the surface of the frozen tuna F. In any case, the freshness determination apparatus 10 is unable to obtain appropriate waveform data, and it is difficult to perform accurate freshness determination of the frozen tuna F. This machine training model may be stored in, for example, the storage unit 13, and in this case, the measurement failure detection unit 124 uses a trained machine training model stored in the storage unit 13.

The measurement failure detection unit 124 receives, from the data acquisition 125, the input of the data of reflective waves reflected by the frozen tuna F after ultrasonic waves with the frequency of 500 MHz are emitted from the ultrasonic probe 30. If the input of data of reflective waves is started, the measurement failure detection unit 124 notifies the result notification unit 123 of the measurement start.

Next, the measurement failure detection unit 124 inputs the acquired data of reflective waves to a held machine training model, and determines whether or not a measurement failure has occurred. That is, the measurement failure detection unit 124 determines whether or not a contact failure of the ultrasonic probe 30 for the target object such as a contact failure, peel-off, dirt, damages to the target object, and the dirt on the target object have occurred. In a case where a measurement failure has not occurred, the measurement failure detection unit 124 outputs the data of reflective waves to the result notification unit 123 and the poor freshness determination unit 126. In contrast to this, in a case where a measurement failure has occurred, the measurement failure detection unit 124 outputs information regarding the measurement failure that has been output from the machine training model, to the result notification unit 123.

The poor freshness determination unit 126 includes a trained machine training model for freshness determination that outputs information regarding poor freshness, using waveform data as an input. The machine training model for freshness determination, a Support Vector Machine (SVMV) or a Neural Network can be used. Further, information regarding poor freshness may be indicated in stages or may be a continuous value. The machine training model for freshness determination included in the poor freshness determination unit 126 is trained using a pair of waveform data of a frozen tuna and a chemical analysis result of the frozen tuna, as training data, for example. Aside from this, the machine training model for freshness determination may be trained using a pair of waveform data of a frozen tuna and a result of determination of freshness of the frozen tuna that has been made by a person, as training data. The machine training model for freshness determination may be stored in the storage unit 13, for example, and in this case, the poor freshness determination unit 126 uses a trained machine training model stored in the storage unit 13.

If a measurement failure has not occurred, the poor freshness determination unit 126 obtains waveform data of reflective waves by receiving, from the measurement failure detection unit 124, the input of the data of reflective waves reflected by the frozen tuna F after ultrasonic waves with the frequency of 500 kHz are emitted from the ultrasonic probe 30. Then, the poor freshness determination unit 126 inputs the waveform data to the trained machine training model for freshness determination, and performs the determination of poor freshness at a measurement position in the frozen tuna F at the time point. That is, the poor freshness determination unit 126 inputs the waveform data obtained by ultrasonic waves with a frequency from 20 kHz to 1 MHz that have been propagated in the frozen tuna F being a frozen target object, to the machine training model, and determines the freshness of the frozen tuna F.

By postmortem rigidity, a Young's modulus indicating the elasticity of a fish body and a viscosity coefficient vary. Both the Young's modulus and the viscosity coefficient are physical property values affecting sound propagation. In the case of poor freshness, due to a change in Young's modulus, the amplitude of reflection from a backbone is larger as compared with a case where a frozen tuna is normal. That is, in the graphs 303 and 304 illustrated in FIG. 5, the vicinity of the largest amplitude indicates a reflective wave from the backbone. Further, in the case of poor freshness, due to a change in viscosity coefficient, reflective waves from a portion deeper than the backbone easily attenuate. In the graphs 303 and 304, a part posterior to the vicinity of the largest amplitude (i.e., right side of the paper surface) indicates a reflective wave from the portion deeper than the backbone. As compared with the graphs 301 and 302, in the graphs 303 and 304, a portion of the largest amplitude of the waveform and a portion with a longer transmission and reception elapsed time have a larger difference. In other words, the amplitude rapidly decreases from the portion of the largest amplitude in a direction in which a transmission and reception elapsed time becomes longer. In this manner, it can be seen that, in the case of poor freshness, as compared with a case where the frozen tuna F is normal, a reflective wave of a portion deeper than the backbone rapidly decreases. Thus, in accordance with the condition, the poor freshness determination unit 126 can determine freshness from waveform data.

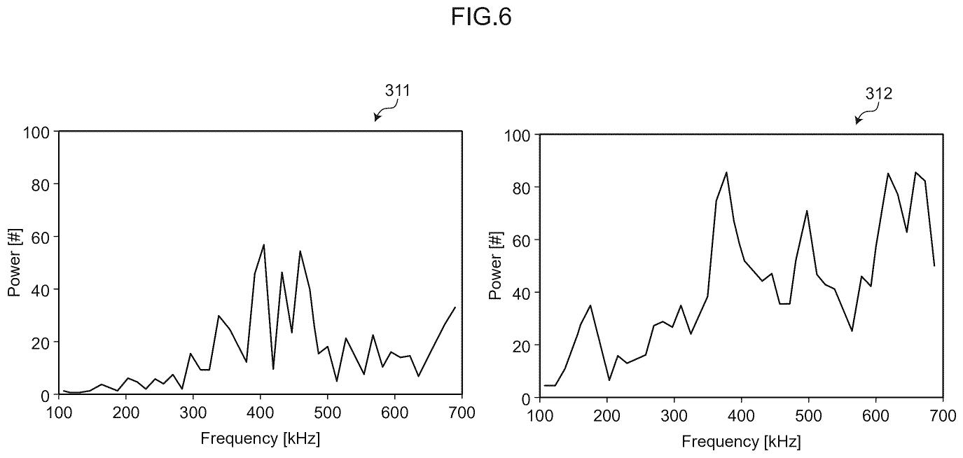

More specifically, the poor freshness determination unit 126 according to the present embodiment determines the freshness of the frozen tuna F by the following method. The poor freshness determination unit 126 calculates a power spectrum by performing Fourier transformation of waveform data of reflective waves. FIG. 6 is a diagram illustrating an example of a power spectrum. A graph 311 in FIG. 6 is a waveform data converted into a power spectrum of a normal frozen tuna F, and a graph 312 is a waveform data converted into a power spectrum of a frozen tuna F with poor freshness. In the graphs 311 and 312, a vertical axis indicates strength and a horizontal axis indicates frequency.

Next, the poor freshness determination unit 126 inputs a waveform data indicated by the calculated power spectrum, to the machine training model, and acquires information regarding poor freshness that is output from the machine training model for poor freshness determination. The power spectrum is data that depends on the frequency, and the influence in intensity of signals to be emitted and a collection range of reflective waves can be suppressed. Thus, by using the power spectrum, the poor freshness determination unit 126 becomes able to utilize even data with a different collection section of a spectrum for training and evaluation of the machine training model for poor freshness determination. Further, by using the power spectrum, the poor freshness determination unit 126 becomes able to obtain constant data from the machine training model for poor freshness determination irrespective of a starting point and an end point of the waveform. That is, in a case where the power spectrum is used, the poor freshness determination unit 126 can easily perform poor freshness determination processing.

The poor freshness determination unit 126 stores a determination result of the freshness of the frozen tuna F at a measurement position at the time into the storage unit 13. At this time, the poor freshness determination unit 126 may notify the result notification unit 123 of a determination result of the freshness of the frozen tuna F at a measurement position at the time.

The result notification unit 123 receives a measurement start notification from the poor freshness determination unit 126. Then, the result notification unit 123 notifies the user P that measurement is being executed, by displaying information for notifying that measurement has been started, on the display device of the input-output unit 11.

In a case where a measurement failure has occurred, the result notification unit 123 receives the input of information regarding the measurement failure from the measurement failure detection unit 124. The information regarding the measurement failure incudes information regarding the occurrence of peel-off, dirt, a contact failure, or the like, for example. Then, the result notification unit 123 notifies the user P of the measurement failure by displaying the information regarding the measurement failure on the display device of the input-output unit 11.

Further, in a case where a measurement failure has not occurred, the result notification unit 123 receives the input of data of reflective waves from the measurement failure detection unit 124. Then, the result notification unit 123 notifies the user P that waveform data is being acquired, by displaying information indicating that the acquisition of waveform data is being performed, on the display device of the input-output unit 11.

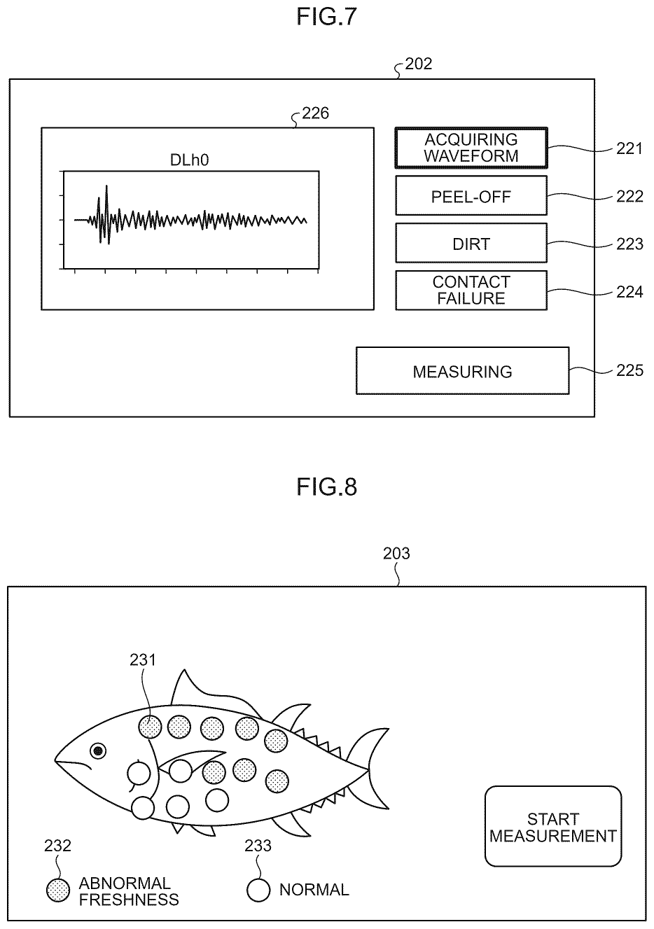

FIG. 7 is a diagram illustrating an example of a measurement in-execution screen. The result notification unit 123 displays a measurement in-execution screen 202 illustrated in FIG. 7, for example, on the display device of the input-output unit 11. If waveform is being acquired, the result notification unit 123 highlights a display field 221 on the measurement in-execution screen 202. At this time, the result notification unit 123 may display information 116 regarding a waveform being acquired, on the measurement in-execution screen 202.

Further, in a case where a measurement failure has occurred, the result notification unit 123 highlights display fields 222 to 224 in accordance with the type of the measurement failure. The result notification unit 123 thereby notifies the user P of information regarding the measurement failure. Further, the poor freshness determination unit 126 may provide a display field 225 indicating that waveform data is being measured, on the measurement in-execution screen 202.

Then, after freshness determination executed by the poor freshness determination unit 126, the result notification unit 123 acquires a determination result of the freshness of the frozen tuna F at a measurement position at the time point from the storage unit 13. Next, the result notification unit 123 notifies the user P of a determination result of the freshness of the frozen tuna F at a measurement position at the time point by displaying the determination result on the display device of the input-output unit 11.

Furthermore, if freshness determination at all measurement positions is completed, the result notification unit 123 receives an inspection completion notification from the measurement position designation unit 121. Next, the result notification unit 123 acquires a determination result of the freshness of the frozen tuna F at each measurement position from the storage unit 13. Then, the result notification unit 123 notifies the user P of a determination result of the freshness of the frozen tuna F at each measurement position by displaying the determination result on the display device of the input-output unit 11. Furthermore, the result notification unit 123 may notify the user P of the evaluation of freshness of the entire frozen tuna F.

FIG. 8 is a diagram illustrating an example of an overall result display screen. After the completion of freshness determination at all measurement positions, the poor freshness determination unit 126 displays an overall result display screen 203 illustrated in FIG. 8, for example, on the display device of the input-output unit 11.

In the present embodiment, as illustrated in FIG. 8, the poor freshness determination unit 126 arranges an image of the frozen tuna F on the overall result display screen 203, and arranges each measurement position on the image of the frozen tuna F. Furthermore, the measurement position designation unit 121 adds a specific pattern to a freshness determination result at each measurement position arranged on the overall result display screen 203, and displays the freshness determination result. In the overall result display screen 203 in FIG. 8, the poor freshness determination unit 126 adds a pattern indicating whether a portion corresponding to each measurement position is poor freshness or normal. Further, the measurement position designation unit 121 arranges display examples 232 and 233 of patterns indicating freshness determination results, on the overall result display screen 203. Further, as described above, the measurement position designation unit 121 may display the evaluation of freshness of the entire frozen tuna F on the overall result display screen 203.

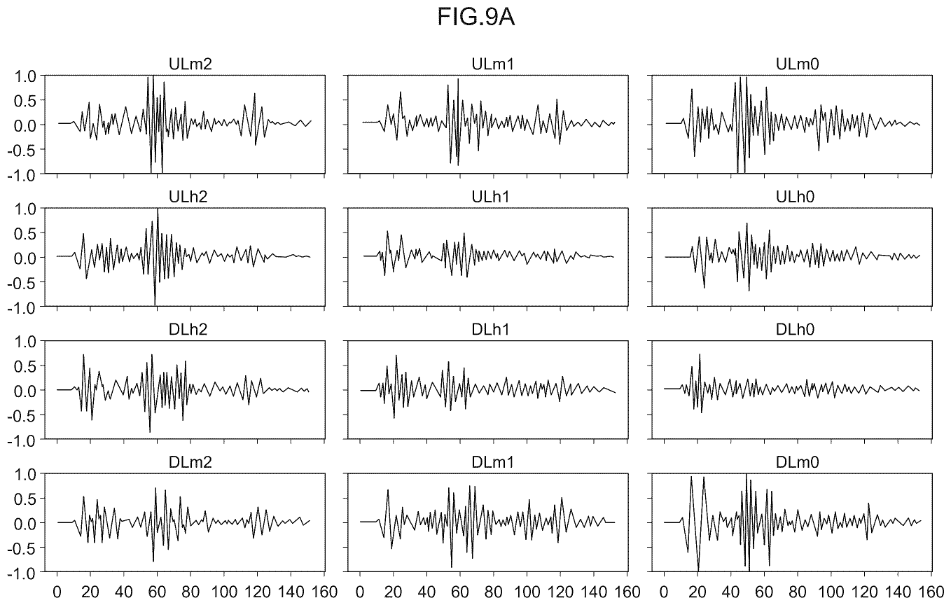

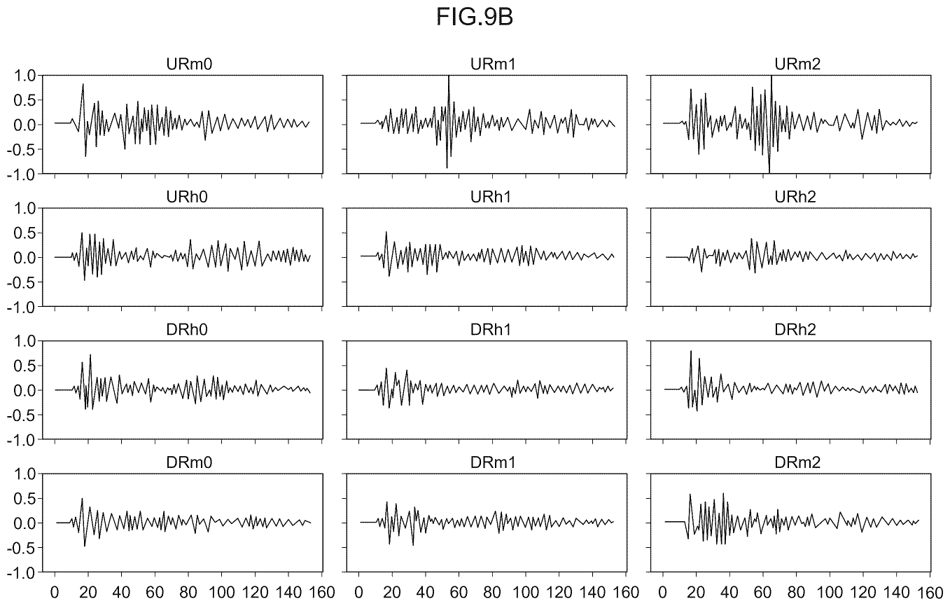

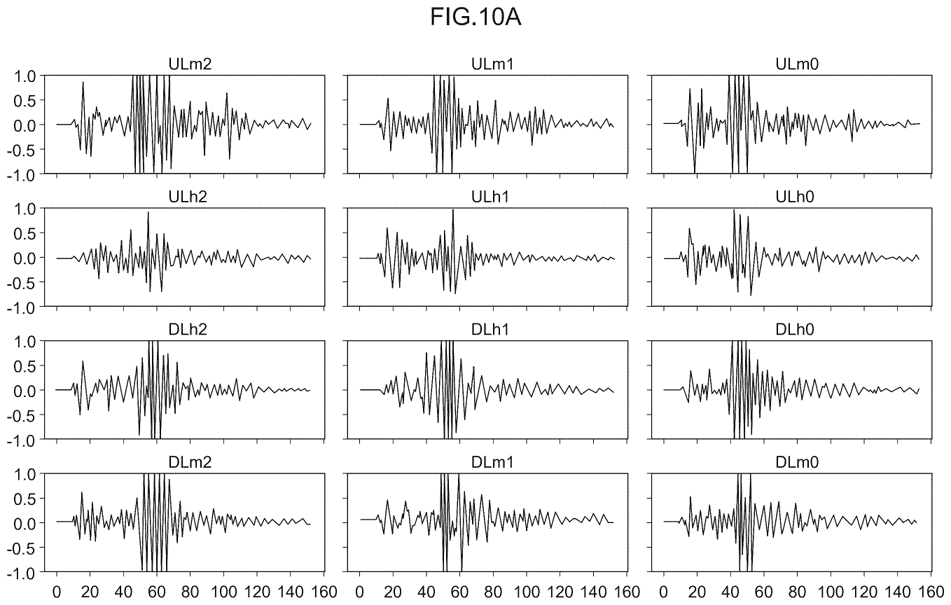

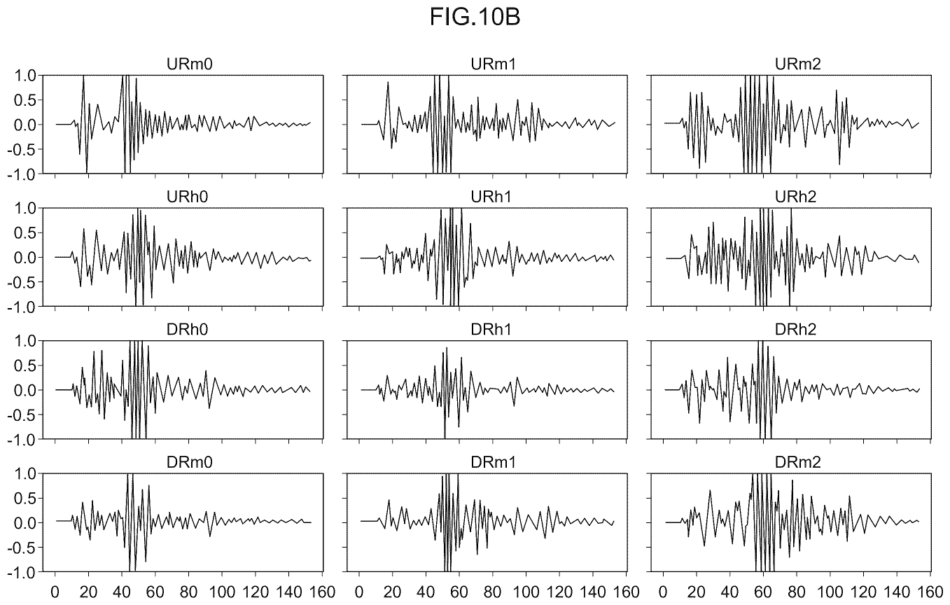

FIG. 9A is a first diagram illustrating a representative example of waveform data of a reflective wave obtained from a normal frozen tuna. FIG. 9B is a second diagram illustrating a representative example of waveform data of a reflective wave obtained from a normal frozen tuna. Further, FIG. 10A is a first diagram illustrating a representative example of waveform data of a reflective wave obtained from a frozen tuna with poor freshness. FIG. 10B is a second diagram illustrating a representative example of waveform data of a reflective wave obtained from a frozen tuna with poor freshness. In graphs in FIGS. 9A, 9B, 10A, and 10B, a horizontal axis indicates an elapsed time of transmission and reception, and a vertical axis indicates an amplitude.

The graphs in FIGS. 10A and 10B apparently differ from the graphs in FIGS. 9A and 9B in that, in the case of poor freshness, the amplitude of a reflective wave from a portion deeper than the backbone rapidly decreases as compared with the amplitude of reflection from the backbone. That is, because a characteristic waveform data of reflective waves exists in the case of poor freshness, by using the machine training model for poor freshness determination that has been trained using these pieces of waveform data, the freshness determination apparatus 10 can distinguish between a normal state and poor freshness, and can accurately perform the determination of freshness.

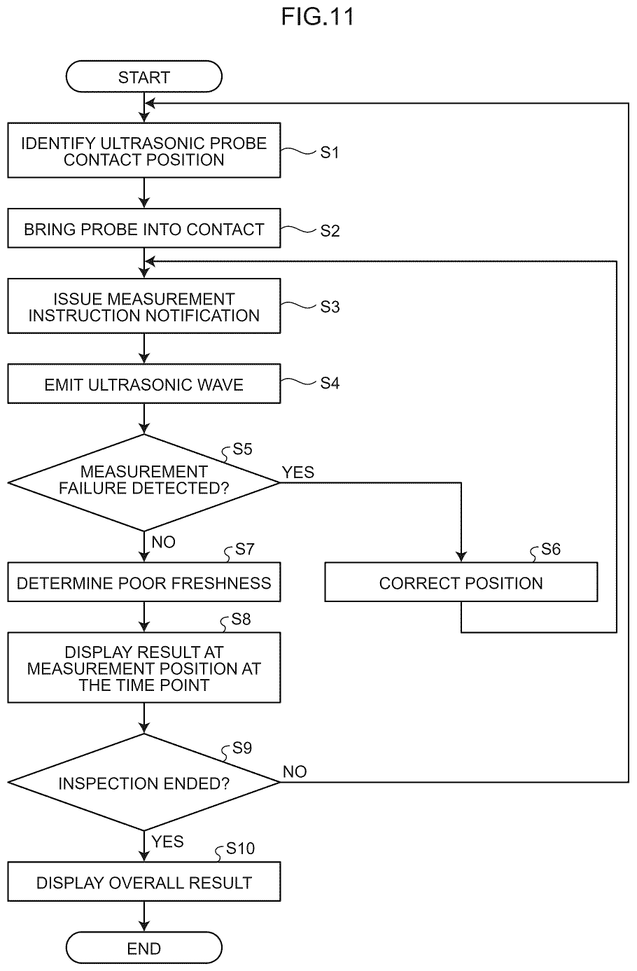

FIG. 11 is a flowchart of freshness determination processing according to an embodiment. Next, a flow of freshness determination processing to be executed by the freshness determination system 1 according to an embodiment will be described with reference to FIG. 11.

The measurement position designation unit 121 selects one measurement position from among a plurality of measurement positions, and determines the selected measurement position as a probe contact position. The measurement position designation unit 121 instructs a probe contact position in the frozen tuna F with which the ultrasonic probe 30 is brought into contact, to the user P by displaying the measurement position designation screen 201 indicating the probe contact position, on the display device of the input-output unit 11 (Step S1).

The user P checks a probe contact position by referring to the measurement position designation screen 201, and brings the ultrasonic probe 30 into contact with the designated probe contact position (Step S2).

The user P issues a measurement start instruction by pressing the measurement start button 213 provided on the measurement position designation screen 201. If the measurement start button 213 provided on the measurement position designation screen 201 is pressed by the user P, the measurement instruction unit 122 receives a measurement start instruction from the input-output unit 11. Then, the measurement instruction unit 122 issues an ultrasonic wave transmission instruction to the ultrasonic inspection apparatus 20 (Step S3).

The ultrasonic inspection apparatus 20 receives an ultrasonic wave transmission instruction from the freshness determination apparatus 10. Then, the ultrasonic inspection apparatus 20 transmits information regarding ultrasonic irradiation settings such as frequency, power, and directions to the ultrasonic probe 30, and causes the ultrasonic probe 30 to emit ultrasonic waves to the frozen tuna F (Step S4). After that, the ultrasonic inspection apparatus 20 acquires a reflected signal of the ultrasonic wave propagated in the frozen tuna F, using the ultrasonic probe 30.

The measurement failure detection unit 124 receives, from the data acquisition 125, the input of the data of reflective waves reflected by the frozen tuna F after the ultrasonic waves are emitted from the ultrasonic probe 30. Then, the measurement failure detection unit 124 inputs the acquired data of reflective waves to a held machine training model, and determines whether or not a measurement failure has been detected (Step S5).

In a case where a measurement failure has been detected (Step S5: Yes), the measurement failure detection unit 124 outputs information regarding the measurement failure to the result notification unit 123. The result notification unit 123 notifies the user P of the measurement failure by displaying the information regarding the measurement failure that has been input from the measurement failure detection unit 124, on the measurement in-execution screen 202 displayed on the display device of the input-output unit 11. Upon receiving the information regarding the measurement failure, the user P corrects the position of the ultrasonic probe 30 in such a manner that measurement can be appropriate performed (Step S6). After that, the freshness determination processing returns to Step S3.

In contrast to this, in a case where a measurement failure has not been detected (Step S5: No), the measurement failure detection unit 124 outputs data of reflective waves to the poor freshness determination unit 126. The poor freshness determination unit 126 obtains waveform data of reflective waves by receiving, from the measurement failure detection unit 124, the input of the data of reflective waves reflected by the frozen tuna F after ultrasonic waves are emitted from the ultrasonic probe 30. Then, the poor freshness determination unit 126 uses the waveform data for the machine training model for freshness determination, and performs the determination of poor freshness at a measurement position in the frozen tuna F at the time point (Step S7).

After freshness determination executed by the poor freshness determination unit 126, the result notification unit 123 acquires a determination result of the freshness of the frozen tuna F at a measurement position at the time point from the storage unit 13. Next, the result notification unit 123 notifies the user P of a determination result of the freshness of the frozen tuna F at a measurement position at the time point by displaying the determination result on the display device of the input-output unit 11 (Step S8).

After the result notification unit 123 has made a notification indicating a result at a measurement position at the time point, the measurement position designation unit 121 determines whether or not the inspection has ended (Step S9). In a case where a measurement position of which freshness determination has not been performed remains, and the inspection has not ended (Step S9: No), the freshness determination processing returns to Step S1.

In contrast to this, in a case where freshness determination at all measurement positions has been completed and the inspection has ended (Step S9: Yes), the result notification unit 123 acquires a determination result of the freshness of the frozen tuna F at each measurement position from the storage unit 13. Then, the result notification unit 123 displays an overall result including a determination result of the freshness of the frozen tuna F at each measurement position, on the display device of the input-output unit 11 (Step S10). The user P can check the overall result and recognize the freshness of the frozen tuna F.

As described above, the freshness determination apparatus according to the present embodiment receives waveform data of reflective waves of ultrasonic waves emitted to a frozen target object, and determines the freshness of the frozen target object using a machine training model. The freshness determination apparatus according to the present embodiment can thereby nondestructively determine the freshness of the frozen target object.

Further, in a case where a measurement failure such as peel-off, dirt, or a contact failure of an ultrasonic probe has occurred, the freshness determination apparatus according to the present embodiment notifies the user of the type of the measurement failure, causes the user to correct a contact position of an ultrasonic probe, performs scanning again, iteratively acquires an ultrasonic waveform, and performs freshness determination. It is accordingly possible to acquire appropriate waveform data, and it becomes possible to accurately determine the freshness of a frozen target object.

### (b) Second Embodiment

Next, the second embodiment will be described. The freshness determination apparatus 10 according to the present embodiment differs from that in the first embodiment in that freshness determination is performed using data included in a predetermined section, among waveform data of reflective waves of ultrasonic waves. The freshness determination apparatus 10 according to the present embodiment is illustrated in FIG. 2. In the following description, the description of operations of components similar to those in the first embodiment will be omitted.

Because the freshness of the frozen tuna F changes not locally but over the entire fish meat of the frozen tuna F, it is preferable to perform freshness determination using reflective waves in a certain section. On the other hand, a waveform of ultrasonic waves attenuates as getting away from the surface of the frozen tuna F. Furthermore, the waveform of an ultrasonic wave after the lapse of long time reflects the influence of multireflection, and might lack information regarding the fish meat. Further, a reflective wave includes information regarding fish meat in a path through which the reflective wave has passed, and freshness determination can be performed based on the information, but there is little information regarding the entire fish meat included in a reflective wave in an early time. From this, it is preferable to use a reflective wave in a predetermined section separated from the surface, in freshness determination.

Furthermore, regarding waveform data, a propagation distance of an ultrasonic wave can be calculated from a sound speed, and it can be seen that a large reflective wave occurs at the position of the backbone. From this, as confirmed from FIGS. 9 and 10, as compared with the normal frozen tuna F, in waveform data of reflective waves of a frozen tuna F with poor freshness, the amplitude at the position of the backbone is larger than amplitudes of other positions, and its characteristic can be said to be rapid decrease of the amplitude from the position of the backbone. It is therefore preferable to use waveform data of reflective waves in a section including the backbone, for freshness determination. Thus, the freshness determination apparatus 10 according to the present embodiment performs freshness determination as follows.

The poor freshness determination unit 126 identifies the position of a reflective wave from the backbone in the acquired waveform data of reflective waves. For example, the poor freshness determination unit 126 may determine a position with the largest amplitude of a reflective wave to be a reflective wave from the backbone, or may determine the center of a section in which an average value of amplitudes in a fixed section is the largest, to be a position of the backbone.

Next, the poor freshness determination unit 126 acquires data in a range of a predetermined distance across the backbone in an irradiation direction of ultrasonic waves among waveform data of reflective waves. For example, the poor freshness determination unit 126 acquires data in a predetermined section anterior to and posterior to the position of the backbone in the irradiation direction of ultrasonic waves among waveform data of reflective waves. Specifically, the poor freshness determination unit 126 acquires data in a section of transmission and reception elapsed times 40 msec before and after the peak of the amplitude, assuming that the peak of the amplitude is the position of the backbone. Then, the poor freshness determination unit 126 determines the freshness of the frozen tuna F using the acquired data in a predetermined section from the position of the backbone.

Here, in the present embodiment, the section before and after 40 msec from the position of the backbone was used, but a section in an irradiation direction of ultrasonic waves is preferably determined in accordance with a target object. Further, the section in the irradiation direction of ultrasonic waves needs not have equal distance from the position of the backbone serving as the center. For example, regarding the poor freshness determination unit 126, the section in the irradiation direction of ultrasonic waves may be calculated in such a manner that the length from the backbone to the rear side, i.e., the length in a travelling direction of ultrasonic waves as seen from the backbone, is shorter than the length in front of the backbone, i.e., the length in the opposite direction to the travelling direction of ultrasonic waves viewed from the backbone.

Furthermore, in the present embodiment, in the scanning, the freshness determination apparatus 10 acquires waveform data of the entire ranges in the frozen tuna F in the irradiation direction of ultrasonic waves, and performs freshness determination using data in a range with a predetermined distance across the backbone in the irradiation direction of ultrasonic waves among the acquired waveform data. Nevertheless, the scanning is not limited to this, and for example, the freshness determination apparatus 10 may cause the ultrasonic inspection apparatus 20 to control scanning in such a manner as to acquire a reflective wave in a range with a predetermined distance across the backbone in the irradiation direction of ultrasonic waves. In this case, the freshness determination apparatus 10 may set a preliminarily-designated position as the position of the backbone, or acquire waveform data once and identify the position of the backbone from the waveform data, and designate the range to be scanned using the identified position of the backbone, in the ultrasonic inspection apparatus 20.

As described above, the freshness determination apparatus according to the present embodiment performs freshness determination of a frozen target object using data in a predetermined range across the backbone in the irradiation direction of ultrasonic waves among waveform data of reflective waves. It is accordingly possible to perform processing of freshness determination by excluding waveform portions of reflective waves that are not important for freshness determination, thereby reducing the processing load. Further, by excluding waveform portions of reflective waves that are not important for freshness determination, the influence of such reflective waves in freshness determination can be reduced, and more accurate freshness determination can be performed.

### (Hardware)

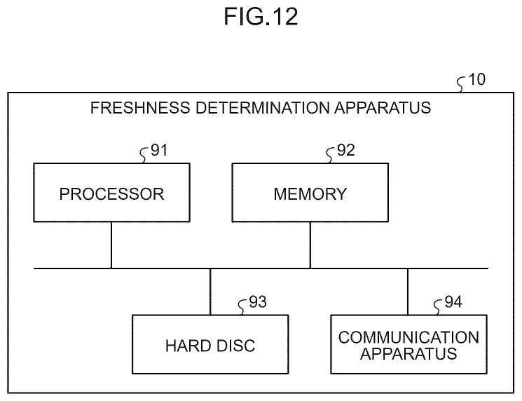

Next, a hardware configuration example of the freshness determination apparatus 10 will be described. FIG. 12 is a diagram of an example of a hardware configuration of a freshness determination apparatus. As illustrated in FIG. 12, the freshness determination apparatus 10 includes a processor 91, a memory 92, a hard disc 93, and a communication apparatus 94. Further, the processor 91 is connected with the memory 92, the hard disc 93, and the communication apparatus 94 via a bus.

The communication apparatus 94 is a communication interface with an external apparatus. For example, the communication apparatus 94 relays communication between the processor 91 and the ultrasonic inspection apparatus 20. Further, the communication apparatus 94 implements the function of the data acquisition 125.

The hard disc 93 is an auxiliary storage device. The hard disc 93 implements the function the storage unit 13 exemplified in FIG. 1. Further, the hard disc 93 stores various programs including a program for implementing the function of the control unit 12 exemplified in FIG. 1.

The processor 91 reads out and loads various programs stored in the hard disc 93, onto the memory 92, and executes the programs. The processor 91 thereby implements the functions of the control unit 12 including the measurement position designation unit 121, the measurement instruction unit 122, the result notification unit 123, the measurement failure detection unit 124, and the poor freshness determination unit 126.

Further, the freshness determination apparatus 10 can also implement functions similar to those in the above-described embodiments, by reading out the above-described programs from a recording medium by a medium reading device, and executing the read above-described programs. In addition, execution of programs is not limited to execution by a specific freshness determination apparatus 10. For example, the present invention can be similarly applied to a case in which another computer or a server executes a program, or a case in which these execute the program in cooperation with each other.

The program can be delivered via a network such as the internet. In addition, the program is recorded on a computer-readable recording medium such as a hard disk, a flexible disk (FD), a CD-ROM, a Magneto-Optical disk (MO), and a Digital Versatile Disc (DVD), and can be executed by being read from the recording medium by a computer.

According to one aspect of a freshness determination program, a freshness determination method, and a freshness determination apparatus disclosed by the present application, an effect of being able to nondestructively determine the freshness of a frozen target object accurately is caused.

What is claimed is:

1. A non-transitory computer-readable recording medium having stored therein a freshness determination program that causes a computer to execute a process comprising:
acquiring waveform data of an ultrasonic wave with a frequency of 1 MHz or less that is propagated in a frozen target object; and
determining freshness of the target object by inputting the waveform data to a machine training model.

2. The non-transitory computer-readable recording medium according to claim 1, wherein the acquiring the waveform data includes acquiring the waveform data including a reflective wave of an ultrasonic wave by a backbone in the target object.

3. The f non-transitory computer-readable recording medium according to claim 2, wherein the acquiring the waveform data includes acquiring waveform data by a reflective wave from a range of a predetermined distance across the backbone in an irradiation direction of an ultrasonic wave.

4. The non-transitory computer-readable recording medium according to claim 1, wherein the process further includes detecting a contact failure of an ultrasonic probe to the target object, damage to the target object, and dirt of the target object based on the waveform data.

5. The non-transitory computer-readable recording medium according to claim 1, wherein the inputting the waveform data to a machine training model includes calculating a power spectrum of the waveform data and inputting the calculated power spectrum to the machine training model.

6. A freshness determination method comprising:
acquiring waveform data of an ultrasonic wave with a frequency of 1 MHz or less that is propagated in a frozen target object; and
determining freshness of the target object by inputting the waveform data to a machine training model, using a processor.

7. A freshness determination apparatus comprising:
a processor configured to:
acquire waveform data of an ultrasonic wave with a frequency of 1 MHz or less that is propagated in a frozen target object, and
determine freshness of the target object by inputting the waveform data to a machine training model.

8. A non-transitory computer-readable recording medium having stored therein a quality determination program that causes a computer to execute a process comprising:
acquiring waveform data of an ultrasonic wave with a frequency of 1 MHz or less that is propagated in a frozen target object; and
determining quality of the target object by inputting the waveform data to a machine training model.

9. A quality determination method comprising:
acquiring waveform data of an ultrasonic wave with a frequency of 1 MHz or less that is propagated in a frozen target object; and
determining quality of the target object by inputting the waveform data to a machine training model, using a processor.

10. A quality determination apparatus comprising:
a processor configured to:
acquire waveform data of an ultrasonic wave with a frequency of 1 MHz or less that is propagated in a frozen target object, and
determine quality of the target object by inputting the waveform data to a machine training model.
